(19) United States
(12) Patent Application Publication
(10) Pub. No.: US 2013/0184245 A1
(43) Pub. Date: Jul. 18, 2013

(54) ENDOMETRIOSIS TREATMENT

(71) Applicant: DRUGTECH CORPORATION, Wilmington, DE (US)

(72) Inventors: Daniel J. Thompson, St. Louis, MO (US); Robert C. Cuca, Glen Carbon, IL (US); Thomas C. Riley, St. Charles, MO (US); Jonathan David Bortz, St. Louis, MO (US)

(73) Assignee: DRUGTECH CORPORATION, Wilmington, DE (US)

(21) Appl. No.: 13/733,213

(22) Filed: Jan. 3, 2013

Related U.S. Application Data
(63) Continuation of application No. 12/105,878, filed on Apr. 18, 2008, now abandoned.
(60) Provisional application No. 60/917,748, filed on May 14, 2007.

(51) Int. Cl. A61K 9/00; A61K 31/58

(52) U.S. Cl. CPC A61K 9/0034; A61K 31/58
USPC 514/176

(57) ABSTRACT

A pharmaceutical composition comprises at least one nonlipoidal internal phase and at least one lipoidal external phase that is bioadhesive to a vaginal mucosal surface, and comprises danazol in an amount of about 3% to about 30% by weight of the composition, wherein upon application of the composition to the vaginal mucosal surface the danazol is released over a period of about 1 to about 10 days. The composition is useful for intravaginal administration to treat a condition such as endometriosis for which danazol is indicated.

### REFERENCE TO RELATED APPLICATIONS

This application is a continuation of application Ser. No. 12/105,878, filed Apr. 18, 2008, which claims the benefit of U.S. Provisional Application Ser. No. 60/917,748, filed on May 14, 2007, the contents of which are incorporated herein by reference in their entirety for all purposes.

### FIELD OF THE INVENTION

The present invention relates to pharmaceutical compositions and use thereof, for example in treatment of inflammatory conditions such as endometriosis, more particularly by intravaginal administration.

### BACKGROUND OF THE INVENTION

Endometriosis is an inflammatory disorder of the female reproductive system, in which a specialized type of tissue that normally forms the endometrium lining the inside wall of the uterus becomes implanted outside the uterus. Implantation of such tissue, referred to herein as endometrial tissue even where not part of the endometrium itself, occurs most frequently in the pelvic region, for example on the fallopian tubes and/or ovaries, on the outer surface of the uterus and on ligaments supporting the uterus, on the lining of the pelvic cavity, or other abdominal sites including bladder, bowel, vagina, cervix and vulva. Endometrial tissue can also implant in abdominal surgical scars and, more rarely, in sites outside the abdominal or pelvic region.

Like normal endometrium, ectopic (i.e., outside the uterus) endometrial tissue responds to hormonal changes during the menstrual cycle, thickening with blood and then breaking down to shed the blood. Blood produced by breakdown of ectopic endometrial tissue cannot exit the body in the normal process of menstruation, and instead it is trapped, causing irritation of surrounding tissues. Trapped blood can lead to growth of cysts which, in turn, can form scar tissue and adhesions that become painful, especially during menstruation. Endometriosis is thus one of the causes of dysmenorrhea (painful periods). It can also result in dyspareunia (pain during sexual intercourse) and infertility; approximately one-third to one-half of all women who report difficulty in becoming pregnant have endometriosis.

It has been estimated that about 5.5 million women in North America, and as many as 70 million women worldwide, are affected by endometriosis. Despite this, the treatment options available are limited.

Analgesics such as nonsteroidal anti-inflammatory drugs (NSAIDs) can be effective in some cases to relieve pain resulting from endometriosis, but they do nothing to address the underlying cause of the pain or other effects of endometriosis such as infertility. Where pain is severe, analgesics may give insufficient relief

Hormonal therapies form the mainstay of endometriosis treatment. Endometrial tissue requires estrogen to grow, and itself produces estrogen. Thus hormonal therapies that lower estrogen levels and/or increase levels of hormones that counteract effects of estrogen can be effective. Oral contraceptives, for example, can reduce or eliminate pain arising from endometriosis. Progestins and androgens (natural or, more commonly, synthetic) interfere with the monthly cycle of endometrial thickening, breakdown and bleeding. The most widely used progestin, medroxyprogesterone, is typically administered as a depot injection. Androgens, including danazol (17α-ethynyl-17β-hydroxy-4-androsteno[2,3-d]-isoxazole) and gestrinone (ethylnorgestrienone), can be administered orally. Hormonal therapy also includes use of drugs that interact with gonadotropin releasing hormone (GnRH), whether as agonists (e.g., nafarelin or leuprolide) or antagonists, and further includes use of aromatase inhibitors that block conversion of androgens such as androstenedione and testosterone to estrogen.

Hormonal therapies administered systemically, for example orally, intranasally or by injection, inevitably result in systemic effects that can significantly restrict their usefulness. For example, side effects of medroxyprogesterone can include weight gain and depression. Side effects of androgens and aromatase inhibitors can include oily skin (often leading to acne), as well as facial hair growth and other masculinization effects such as a deepening of the voice. GnRH agonists and antagonists can create an artificial menopause and, with it, side effects such as hot flashes and vaginal dryness. The anti-estrogen nature of systemic hormone therapies typically inhibits ovulation and conception, thus for women seeking to overcome fertility problems arising from endometriosis such therapies may not provide the desired outcome.

Danazol, when administered orally, is typically prescribed at a daily dose of 200 to 800 mg for a treatment period of 3-6 months, occasionally extended to 9 months. Because of its androgenic side effects and a tendency to adversely affect blood lipid levels, danazol has not typically been the first choice of treatment for endometriosis. However, a significant advance in the art occurred with development of pharmaceutical compositions and delivery systems for intravaginal administration of androgens such as gestrinone and danazol.

For example, Coutinho & Azadian-Boulanger (1988) Fertility and Sterility 49(3):418-422 reported that gestrinone administered by a vaginal route to women with endometriosis was equally effective to oral gestrinone therapy in reducing dyspareunia and dysmenorrhea, but that side effects such as seborrhea, acne and weight gain were significantly lower with vaginal administration.

Igarashi (1990) Asia-Oceania J. Obstet. Gyn. 16(1):1-12 reported that a vaginal ring containing 2000 to 3500 mg danazol not only reduced dysmenorrhea and pelvic endometriosis in 35 infertile women, but enabled conception in 13 of these women while the ring was in place.

Igarashi et al. (1997) Acta Obstet. Gyn. Scand. 76 (Suppl. 167 part 3):30 reported that topical administration of danazol, by intravaginal insertion of a vaginal ring containing 1000 mg danazol, was very effective in treating pelvic endometriosis. Serum danazol levels with danazol 400 mg/day per os reportedly exceeded 100 ng/ml, but were always undetectable with topical danazol administration.

Igarashi et al. (1998) Human Reproduction 13(7):1952-1956 reported a study in which danazol was administered via the vagina, using a vaginal ring drug delivery system containing 1500 mg danazol. Dysmenorrhea was reportedly eliminated within 3 months. Ovulation and conception reportedly occurred in 17 out of 31 infertile women with deeply infiltrating endometriosis, and general side-effects common in oral danazol therapy were reportedly not observed. Serum danazol concentrations were said to be undetectable with vaginal danazol therapy, but high during oral therapy with danazol at 400 mg/day.

Mizutani et al. (1995) Fertility and Sterility 63(6):1184-1189 compared effect of danazol administration per os (400 mg/day) or as a vaginal suppository (100 mg/day) on danazol concentrations in the ovary, uterus and serum. Concentrations in the ovary and uterus with vaginal administration were reported to be comparable to those with oral administration, but serum danazol concentration was reportedly much lower in the case of vaginal administration.

More recently, Dmowski & Janicki (2004) Obstet. Gyn. 103(4 Suppl.):60S reported a clinical trial evaluating safety, efficacy and pharmacokinetics of intravaginal danazol therapy in 22 women with endometriosis and chronic pelvic pain symptoms. The therapy was reported to be effective for management of chronic pelvic pain secondary to pelvic endometriosis, to be well tolerated, and to demonstrate a favorable side effect profile with a paucity of systemic androgen-like effects. Peripheral levels of danazol were reportedly undetectable.

Norton (2004) Ob/Gyn News Oct. 1, 2004 (http://www.findarticles.com/p/articles/mi_mOCYD/is_{—}19_{—}39/ai_n6260169), reporting on a poster presentation by Janicki, stated that a novel intravaginal danazol formulation (a gel preparation by FemmePharma Inc. said to adhere to the vaginal wall) significantly reduced chronic pelvic pain in a study of women with endometriosis. It was reported that “[m]ost importantly, there was a lack of systemic androgenic side effects, and low to undetectable plasma levels of danazol, indicating that the drug was acting locally.” All of the women in the study reportedly retained their menses and ovulation. Danazol dosage was 100 to 200 mg/day, administered intravaginally with an applicator.

U.S. Pat. No. 4,997,653 to Igarashi relates inter alia to a vaginal ring preparation comprising a matrix base of a polymeric material such as polydimethylsiloxane having 50 to 4000 mg danazol retained therein, and use of such a preparation for treatment of endometriosis.

European Patent Publication No. 0 501 056 of Igarashi mentions a report of intravaginal administration of 500 to 600 mg, once weekly, presented at the 12th Endometriosis Research Meeting in Kyoto in 1991. The '056 publication goes on to propose a powder, tablet, suppository, capsule or liquid composition comprising 0.5 to 100 mg danazol for vaginal administration. For treatment of endometriosis, a dosage of 0.5 to 20 mg danazol, administered intravaginally once every 1 to 4 days, is proposed. Daily administration of 5 mg danazol in suppository form is stated therein to provide remission of, inter alia, dyspareunia and dysmenorrhea.

U.S. Patent Application Publication No. 2002/0150605 to Yui et al. relates to a pharmaceutical preparation for treatment of gynecological diseases including endometriosis by, for example, intravaginal administration. The preparation comprises a drug, for example danazol, and a biodegradable polymer comprising a chemically modified hyaluronic acid or a salt thereof, and is said to be suitable for sustained release of the drug.

U.S. Pat. No. 5,993,856 to Ragavan & DiPiano relates to formulations for topical or local delivery of a drug, for example danazol, by administration to the female reproductive system. Such administration is said to provide relatively high blood levels of the drug in the region to be treated in the substantial absence of systemic levels of the drug which might cause side effects. The formulations contain drug micro- or nanoparticles and can reportedly be applied as a dried powder, a liquid suspension or dispersion, or as a topical ointment, cream, lotion, foam or suppository. An excipient or polymer in such a formulation can reportedly be used to manipulate release rate of the drug and to increase adhesion to the affected region. Specifically exemplified is a danazol 1 mg/50 μl (2% weight/volume) composition prepared by levigating danazol in K-Y® Jelly, a commercial hydroxyethylcellulose gel product.

Thompson & Levinson (2002), Drug Delivery Systems & Sciences 2(1), 17-19, describe a bioadhesive topical drug delivery system known therein as the VagiSite system as a high internal phase ratio water-in-oil emulsion system, providing a delivery platform for administration of active drug entities in the vaginal cavity.

U.S. Patent Application Publication No. 2003/0180366 of Kirschner et al. relates to an essentially pH neutral emulsion formulation for vaginal drug delivery, having an internal acidic buffered water-soluble phase and an external water-insoluble phase or film. The internal phase can contain a micronized drug having a particle size of 0.1 to less than 60 μm. Among drugs deliverable by such a formulation are androgenic substances, for example danazol, testosterone or a combination thereof. Among disorders treatable by such a formulation are all forms of endometriosis. Upon administration, the drug is said to be released over a period of about 0.1 hour to about 168 hours (one week), for example about 0.1 hour to about 72 hours (three days). Formulations exemplified include a composition comprising 0.75% metronidazole and a composition comprising 2.8% clindamycin phosphate.

It would be desirable to provide a vaginal cream composition that is capable of delivering danazol in an amount effective for treatment of endometriosis, i.e. an amount of up to about 150 mg/day, when administered intravaginally at a once daily or lower frequency.

### SUMMARY OF THE INVENTION

There is now provided a pharmaceutical composition comprising at least one nonlipoidal internal phase and at least one lipoidal external phase that is bioadhesive to a vaginal mucosal surface, and comprising danazol in an amount of about 1% to about 30%, more typically about 3% to about 30%, by weight of the composition, wherein upon application of the composition to the vaginal mucosal surface the danazol is released over a period of about 1 to about 10 days.

There is further provided a pharmaceutical composition comprising at least one nonlipoidal internal phase and at least one lipoidal external phase that is bioadhesive to a vaginal mucosal surface, and comprising about 20% danazol, wherein the internal phase comprises about 45% water and about 20% sorbitol and the external phase comprises about 8% mineral oil, about 2.8% polyglyceryl-3-oleate, about 2.8% glyceryl monoisostearate, about 0.5% microcrystalline wax and about 1% hydrophobic silica, all percentages being by weight of the composition as a whole; or a composition substantially bioequivalent thereto when administered intravaginally.

There is still further provided a process for preparing a danazol intravaginal cream composition, comprising
(a) admixing one or more hydrophilic ingredients including at least one pharmaceutically acceptable polyol with water to provide an aqueous phase;
(b) admixing one or more hydrophobic ingredients including at least one emulsifying agent and optionally at least one viscosity modulating agent with at least one pharmaceutically acceptable oil to provide a lipoidal phase;
(c) admixing a first portion of the aqueous phase to the lipoidal phase to form an intermediate dispersion;
(d) adding micronized particulate danazol to the intermediate dispersion with mixing to provide a uniform premix; and
(e) admixing the balance of the aqueous phase to the premix with mixing to provide the finished composition;
   wherein the ingredients and relative amounts thereof are selected to provide a finished composition having the lipoidal phase continuous and external to the aqueous phase; and
   wherein the danazol is added in an amount of about 1% to about 30%, more typically about 3% to about 30%, by weight of the finished composition.

There is still further provided a vaginal danazol delivery system comprising (a) a pharmaceutical composition comprising at least one nonlipoidal internal phase and at least one lipoidal external phase that is bioadhesive to a vaginal mucosal surface, and comprising danazol in an amount of about 1% to about 30%, more typically about 3% to about 30%, by weight of the composition, wherein upon application of the composition to the vaginal mucosal surface the danazol is released over a period of about 1 to about 10 days; and (b) an applicator.

There is still further provided a method for treating a condition in a female subject for which danazol is indicated, comprising administering intravaginally to the subject a therapeutically effective amount of a pharmaceutical composition that comprises at least one nonlipoidal internal phase and at least one lipoidal external phase that is bioadhesive to a vaginal mucosal surface, and comprising danazol in an amount of about 1% to about 30%, more typically about 3% to about 30%, by weight of the composition, wherein upon application of the composition to the vaginal mucosal surface the danazol is released over a period of about 1 to about 10 days.

In one embodiment the condition treated by such a method is endometriosis.

There is still further provided a contraceptive method, comprising administering intravaginally to a female subject a contraceptively effective amount of a pharmaceutical composition that comprises at least one nonlipoidal internal phase and at least one lipoidal external phase that is bioadhesive to a vaginal mucosal surface, and comprising danazol in an amount of about 1% to about 30%, more typically about 3% to about 30%, by weight of the composition, wherein upon application of the composition to the vaginal mucosal surface the danazol is released over a period of about 1 to about 10 days.

There is still further provided a pre-operative endometrial thinning method, comprising administering intravaginally to a female subject prior to surgery an endometrial thinning effective amount of a pharmaceutical composition that comprises at least one nonlipoidal internal phase and at least one lipoidal external phase that is bioadhesive to a vaginal mucosal surface, and comprising danazol in an amount of about 1% to about 30%, more typically about 3% to about 30%, by weight of the composition, wherein upon application of the composition to the vaginal mucosal surface the danazol is released over a period of about 1 to about 10 days.

Additional embodiments are described in the detailed description that follows.

### DETAILED DESCRIPTION

A composition of the invention is a water-in-oil emulsion, for example of a type generally described in any of the patents and publications individually cited below and incorporated herein by reference.

U.S. Pat. No. 4,551,148 to Riley et al.

U.S. Pat. No. 5,266,329 to Riley.

Above-cited U.S. Patent Application Publication No. 2003/0180366.

U.S. Patent Application Publication No. 2005/0095245 of Riley et al.

Compositions useful herein include creams and dosage forms such as suppositories which are solid at room temperature but melt or soften at body temperature to form a cream. The term “cream” herein refers to a viscous spreadable semi-solid composition having an aqueous phase and a lipoidal phase, generally stabilized by means of one or more emulsifying agents. Conventional vaginal creams are oil-in-water emulsions having a continuous aqueous phase and a discontinuous or disperse lipoidal phase, wherein an active agent is typically solubilized or dispersed in the continuous phase.

However, presence of an active agent in the continuous nonlipoidal phase of an oil-in-water emulsion permits immediate contact of the active agent with the vaginal mucosal surface to which the composition is applied, but also permits dilution, rinsing and leakage of the composition from this surface, which is not only offensive or inconvenient for the patient but can reduce the contact time with the surface. An oil-in-water emulsion therefore must typically be administered at relatively high frequency to provide a clinically acceptable response. Such frequent application increases the potential for systemic delivery of the active agent, and thereby increases the potential for adverse side-effects, and also increases likelihood of local tissue irritation.

By contrast, a vaginal cream of the present invention is a water-in-oil emulsion that comprises at least one nonlipoidal (typically aqueous) internal phase and at least one, typically continuous, lipoidal external phase. The lipoidal external phase is bioadhesive to the vaginal mucosal surface.

Conventional vaginal creams commonly require application at bedtime to take advantage of a supine position of the patient for several hours, which can help to retain the cream within the vaginal cavity. The bioadhesive property and consequently enhanced vaginal retention of a vaginal cream of the invention can enable application at any convenient time of day. Furthermore, where the composition is formulated for sustained release of the active agent, in the present instance danazol, over a period greater than one day, the composition can be retained in the vaginal cavity for the duration of the release period. A particular advantage is that the composition can resist washout or leakage even during menses, thus therapy does not have to be interrupted. It is noted in this regard that vaginal administration of danazol typically does not inhibit menstruation in the way that oral administration does.

Compositions useful herein typically have a viscosity of about 5,000 to about 750,000 centipoise, for example about 100,000 to about 650,000 centipoise or about 350,000 to about 550,000 centipoise.

Notwithstanding the proposal in above-cited European Patent Publication No. 0 501 056 to treat endometriosis by intravaginal administration of 0.5 to 20 mg danazol every 1 to 4 days, it is presently believed that higher doses, especially where administration frequency is once to twice per week, will provide superior results without excessive systemic delivery of the drug. Thus, illustratively, a dose of about 30 to about 150 mg/day can be provided by twice-weekly administration of about 100 to about 500 mg, or weekly administration of about 200 to about 1000 mg, in a form of a composition of the present invention having an appropriate danazol release rate.

A vaginal cream is conveniently administered in an amount of about 0.5 g to about 10 g, although greater or lesser amounts can be useful in particular circumstances. Excessively small amounts can be impracticable as a result of difficulty in ensuring even application to the vaginal mucosal surface; excessively large amounts can exceed the capacity of the vaginal mucosal surface to retain the cream, resulting in leakage and consequent underdosing. In practice, amounts of about 1 g to about 6 g, for example about 1.5 g to about 5 g, are most convenient. For purposes of illustration only, if a 3.3 g amount is administered twice weekly to provide a danazol dose of about 100 to about 500 mg, the cream requires to contain about 3% to about 15% by weight danazol. Similarly, if a 1.7 g amount is administered twice weekly to provide a danazol dose of about 100 to about 500 mg, or a 3.3 g amount is administered weekly to provide a danazol dose of about 200 to about 1000 mg, the cream requires to contain about 6% to about 30% by weight danazol.

Thus in various embodiments of the invention, a vaginal cream composition as described herein comprises danazol in an amount of about 1% to about 30%, more typically about 2% to about 30% or about 3% to about 30%, for example about 5% to about 25% or about 10% to about 20%, by weight of the composition.

Some of these embodiments have relatively high concentrations of danazol (for example at least about 3%, at least about 5% or at least about 10% by weight) by comparison with vaginal compositions in the art. Furthermore, it has now been found that preparation of a vaginal cream having such high concentrations of danazol is a significant challenge, due in part, it is believed, to the poor solubility of danazol in both polar and non-polar solvents.

While a vaginal cream composition of the invention can have a danazol release period, upon application to a vaginal mucosal surface, of about 1 to about 10 days, i.e., a property consistent with an administration frequency of about 1 to about 10 days, in more particular embodiments the release period is about 2 to about 8 days or about 3 to about 7 days, i.e., a property consistent with an administration frequency of once to twice weekly. In an illustrative embodiment the danazol is present in an amount of about 5% to about 25% by weight of the composition, and upon application of the composition to the vaginal mucosal surface, the danazol is released over a period of about 2 to about 8 days.

The relative weights of the internal (nonlipoidal) phase and the external (lipoidal) phase of the composition are not critical, but in general it will be found suitable to prepare the composition with an internal/external weight ratio of about 50:50 to about 85:15, for example about 60:40 to about 80:20. Danazol can be present in either or both of the internal and external phases, but typically at least a substantial part, for example at least about 50%, of the danazol is present in the internal phase.

The bioadhesive property of a composition of the present invention is believed, without being bound by theory, to reside at least in part in the lipoidal nature of the external phase, which repels moisture and thereby resists dilution and removal by normal vaginal secretion. It is further believed, again without being bound by theory, that the lipoidal external phase serves to sequester the internal nonlipoidal phase; where the active agent (in the present instance danazol) is present partly or wholly in the internal phase, the active agent payload is likewise sequestered, allowing for release of the active agent to be metered slowly over time.

The bioadhesive and controlled or sustained release properties of a composition embodying a vaginal delivery system known as the Site Release® (SR) system useful herein have been demonstrated in studies summarized by Merabet et al. (2005), Expert Opin. Drug Deliv. 2(4):769-777, incorporated herein by reference.

Danazol is a highly insoluble compound and is typically present at least in major part in solid particulate form. To ensure adequate and uniform dispersion in the composition as well as to enable adequate release properties, it is generally desirable that the danazol be in micronized particulate form, i.e., having an average particle size of about 1 to about 20 μm, for example about 2 to about 15 μm or about 3 to about 10 μm or in nanoparticulate form, i.e., having an average particle size of less than about 1 μm, for example about 0.1 to about 1 μm.

Illustratively, the internal phase is aqueous and comprises at least one pharmaceutically acceptable polyol. Polyols useful herein include without limitation glycerol, glycols such as polyethylene glycols and propylene glycol, and sugar alcohols such as erythritol, lactitol, maltitol, mannitol, sorbitol and xylitol. More than one such polyol can be present. In a particular embodiment, the at least one polyol comprises sorbitol. As propylene glycol can be irritant to the vagina, it is generally desirable that the composition comprise no vaginal irritant amount of propylene glycol; for example the composition can be free or substantially free of propylene glycol.

Generally the internal phase has high osmotic pressure. The internal phase can itself be monophasic, biphasic or multiphasic, taking the form, for example, of a solution, suspension, emulsion or combination thereof. The internal phase optionally comprises one or more suspended solids, emulsifying and/or dispersing agents, osmotic enhancers, extenders, diluents, buffering agents, chelating agents such as edetate disodium, preservatives, fragrances, colors, or other materials.

Optionally, the internal phase is acid buffered to an internal pH of about 2.0 to about 6.0, for example about 2.5 to about 5.5 or about 3.5 to about 5.0. In one embodiment the internal phase is acid buffered to an internal pH that is substantially optimal to the vaginal environment, i.e., a pH that does not cause substantial irritation, itching or other discomfort and/or renders the vaginal environment less hospitable to common pathogens including fungal and bacterial pathogens. Typically such a pH is about 4.0 to about 5.0, for example approximately 4.5.

The external lipoidal phase is typically continuous. The term “lipoidal” herein can pertain to any of a group of organic compounds including neutral fats, fatty acids, waxes, phosphatides, petrolatum, fatty acid esters of monoprotic alcohols, mineral oils, etc., having the following properties: insoluble in water; soluble in alcohol, ether, chloroform or other fat solvents; and exhibiting a greasy feel. Examples of suitable pharmaceutically acceptable oils are mineral oils having viscosity of about 5.6 to about 68.7 centistokes, for example about 25 to about 65 centistokes, and vegetable oils such as coconut, palm kernel, cocoa butter, cottonseed, peanut, olive, palm, sunflower, sesame, corn, safflower, rapeseed (canola) and soybean oils and fractionated liquid triglycerides of naturally derived short-chain fatty acids.

The term “lipoidal” can also pertain to amphiphilic compounds, including for example natural and synthetic phospholipids. Suitable phospholipids can include, for example phosphatidylcholine esters such as dioleoylphosphatidylcholine, dimyristoyl-phosphatidylcholine, dipentadecanoylphosphatidylcholine, dipalmitoylphosphatidyl-choline (DPPC) and distearoylphosphatidylcholine (DSPC); phosphatidylethanolamine esters such as dioleoylphosphatidylethanolamine and dipalmitoylphosphatidylethanol-amine (DPPE); phosphatidylserine; phosphatidylglycerol; phosphatidylinositol; etc.

Amphiphilic compounds other than phospholipids can also act, optionally together with a phospholipid, as emulsifying agents in a composition of the invention. Any pharmaceutically acceptable emulsifying agent or combination thereof can be used, including without limitation medium and long chain monoglycerides and diglycerides, such as glyceryl monooleate, glyceryl monostearate, glyceryl monoisostearate and glyceryl monopalmitate, polyglyceryl esters of fatty acids, such as polyglyceryl-3 oleate, and polyethylene glycol esters and diesters of fatty acids, such as PEG-30 dipolyhydroxystearate. Emulsifying agents soluble in the external phase are generally preferred. In a particular embodiment, the external phase comprises glyceryl monoisostearate, polyglyceryl-3-oleate or both. Where both are present, the weight ratio of polyglyceryl-3-oleate to glyceryl monoisostearate is illustratively about 1:5 to about 5:1, for example about 1:2 to about 2:1, in a particular embodiment about 1:1. Certain emulsifying agents can also function as emollients in the composition. Additional emollient materials that can be used include propylene glycol esters of fatty acids, and lower alkyl esters of fatty acids such as isopropyl myristate, isopropyl palmitate and methyl oleate.

Preservatives such as parabens, for example methylparaben, propylparaben or both, can optionally be included in the external phase.

Viscosity modulating agents are optionally present in the internal and/or external phases. Optional ingredients that can increase viscosity, among other properties, include microcrystalline wax, colloidal silica, and various pharmaceutically acceptable polymers including polysaccharides, cellulosic polymers such as carboxymethylcellulose, methylcellulose, hydroxypropylmethylcellulose, etc., polyethylene glycol, acrylate polymers and the like. In one embodiment the external phase comprises at least one pharmaceutically acceptable viscosity modulating agent, for example microcrystalline wax, hydrophobic silica or both.

Expressed as percentage by weight of the composition as a whole, a vaginal cream of the invention can illustratively comprise:
danazol, about 1% to about 30%, more typically about 3% to about 30%;
water, about 30% to about 75%;
polyol, e.g., sorbitol, about 10% to about 30%;
chelating agent, e.g., edetate disodium, zero to about 0.25%;
oil, e.g., mineral oil, about 5% to about 15%;
one or more emulsifying agents, e.g., combination of polyglyceryl-3-oleate and glyceryl monoisostearate, about 2% to about 10% total;
microcrystalline wax, zero to about 2%;
hydrophobic silica, zero to about 5%;
methylparaben, zero to about 0.5%; and
propylparaben, zero to about 0.25%.

A specific vaginal cream composition described in the Examples hereinbelow comprises at least one nonlipoidal internal phase and at least one lipoidal external phase that is bioadhesive to a vaginal mucosal surface, and comprises about 20% by weight danazol. The internal phase comprises about 45% water and about 20% sorbitol, and the external phase comprises about 8% mineral oil, about 2.8% polyglyceryl-3-oleate, about 2.8% glyceryl monoisostearate, about 0.5% microcrystalline wax and about 1% hydrophobic silica, all percentages being by weight of the composition as a whole. Other optional ingredients can be present in small amounts.

This composition is believed to provide a particular danazol release profile that is well suited to once to twice weekly intravaginal administration for treatment of endometriosis, and a particular pharmacokinetic (PK) profile characterized by plasma levels of danazol that do not exceed a threshold level above which systemic side effects are prevalent. In one embodiment of the invention, there is therefore provided a composition as described immediately above, or a composition substantially bioequivalent thereto when administered intravaginally. The term “substantially bioequivalent” in relation to a first and second composition herein means that the values of the PK parameters Cₘₐₓ and AUC (for example AUC₀₋₂₄ or AUC_{0-∞}) for danazol from the second composition, administered by the same route and in the same danazol dose, are not less than about 80% and not greater than about 125% of the corresponding values for danazol from the first composition. Bioequivalence can readily be determined by one of skill in the art by conducting a PK study according to standard protocols. Bioequivalence typically requires that the first and second compositions have a substantially similar danazol release profile, as measured in vivo or in an in vitro assay system generally accepted in the art as a model for release at a mucosal surface such as a vaginal mucosal surface.

The present invention further provides a process for preparing a danazol vaginal cream composition as described herein. It will be understood that the present compositions can be made by one of skill in the art by processes that may vary from that described below, without removing such compositions from the scope of the invention.

The process comprises:
(a) admixing one or more hydrophilic ingredients including at least one pharmaceutically acceptable polyol with water to provide an aqueous phase;
(b) admixing one or more hydrophobic ingredients including at least one emulsifying agent and optionally at least one viscosity modulating agent with at least one pharmaceutically acceptable oil to provide a lipoidal phase;
(c) admixing a first portion of the aqueous phase to the lipoidal phase to form an intermediate dispersion;
(d) adding micronized particulate danazol to the intermediate dispersion with mixing to provide a uniform premix; and
(e) admixing the balance of the aqueous phase to the premix with mixing to provide the finished composition;
   wherein the ingredients and relative amounts thereof are selected to provide a finished composition having the lipoidal phase continuous and external to the aqueous phase; and wherein the danazol is added in an amount of about 1% to about 30%, more typically about 2% to about 30% or about 3% to about 30%, by weight of the finished composition.

The first portion of the aqueous phase, added in step (c), can be any amount less than 100%, for example about 10% to about 90% or about 20% to about 80%, by weight of all of the aqueous phase used in preparation of the finished composition. However, it will generally be found advantageous to use about 30% to about 70%, for example about 40% to about 60% by weight, or approximately one-half of the entire aqueous phase as the first portion.

It has been found that by a two-step addition of the aqueous phase, before and after addition of the danazol, it is possible to prepare compositions having the relatively high danazol loading (for example about 3% to about 30% by weight) required by some embodiments of the present invention.

Temperature during mixing is not narrowly critical, but it will be found advantageous to mix at a temperature at which the ingredients flow readily under the mixing shear provided. Where a relatively high melting point ingredient such as microcrystalline wax is to be included in the lipoidal phase, heat can be added in step (b), for example to provide a mixing temperature of about 50° C. to about 85° C., for example about 60° C. to about 80° C. or about 70° C. to about 75° C. If hydrophobic silica as well as microcrystalline wax is to be included in the lipoidal phase, it can be advantageous to cool the mixture prepared in step (b), for example to about 25° C. to about 45° C., for example about 30° C. to about 40° C., prior to adding the hydrophobic silica. Other steps in the process can be conducted at any convenient temperature, but step (a) will normally be conducted at a temperature from ambient (e.g., about 20° C.) to about 40° C., and steps (c)-(e) will normally be conducted at a temperature from ambient to about 30° C., for example approximately 25° C.

Any conventional mixing equipment can be used, for example an appropriately sized stainless steel vessel equipped with an overhead mixer. It may be found advantageous to pass the lipoidal phase prepared in step (b), after addition of hydrophobic silica if any, through an appropriately sized mill or disperser prior to admixing with the aqueous phase in step (c).

The micronized danazol used in preparing the composition advantageously has an average particle size of about 1 to about 20 μm, for example about 2 to about 15 μm or about 3 to about 10 μm. Danazol of relatively uniform particle size generally provides the most efficient process and uniform product. If necessary, the danazol can be pre-milled and/or screened to reduce particle size variability before adding to the composition.

Selection and amounts of ingredients can be as set forth above in description of compositions of the invention. Thus, for example, the weight ratio of aqueous to lipoidal phases can illustratively be about 50:50 to about 85:15, for example about 60:40 to about 80:20. Amounts used in the process, per 100 parts by weight of the finished composition, can illustratively be as follows:
danazol, about 1 to about 30, more typically about 3 to about 30, parts by weight;
water, about 30 to about 75 parts by weight;
polyol, e.g., sorbitol, about 10 to about 30 parts by weight;
chelating agent, e.g., edetate disodium, zero to about 0.25 parts by weight;
oil, e.g., mineral oil, about 5 to about 15 parts by weight;
one or more emulsifying agents, e.g., combination of polyglyceryl-3-oleate and glyceryl monoisostearate, about 2 to about 10 parts by weight total;
microcrystalline wax, zero to about 2 parts by weight;
hydrophobic silica, zero to about 5 parts by weight;
methylparaben, zero to about 0.5 parts by weight; and
propylparaben, zero to about 0.25 parts by weight.

The amount of water indicated above will be understood to include any water added as a solvent or diluent for other ingredients of the aqueous phase. For example, it will normally be convenient to add sorbitol in the form of an aqueous solution, e.g., 65% by weight sorbitol solution; amounts of sorbitol indicated herein are expressed as sorbitol per se, not as sorbitol solution, except where otherwise expressly stated.

In a particular embodiment amounts of ingredients are used to provide a finished composition comprising about 20% by weight danazol, about 45% by weight water, about 20% by weight sorbitol, about 8% by weight mineral oil, about 2.8% by weight polyglyceryl-3-oleate, about 2.8% by weight glyceryl monoisostearate, about 0.5% by weight microcrystalline wax and about 1% by weight hydrophobic silica.

A vaginal cream composition prepared by a process as described above is a further embodiment of the invention.

In yet a further embodiment of the invention, a vaginal danazol delivery system is provided. The delivery system comprises (a) a pharmaceutical composition comprising at least one nonlipoidal internal phase and at least one lipoidal external phase that is bioadhesive to a vaginal mucosal surface, and comprising danazol in an amount of about 1% to about 30%, more typically about 2% to about 30% or about 3% to about 30%, by weight of the composition, wherein upon application of the composition to the vaginal mucosal surface the danazol is released over a period of about 1 to about 10 days; and (b) an applicator.

The composition can be contained in a reservoir that is separate from or integral with the applicator. One or more unit dosage amounts (e.g., each of about 0.5 g to about 10 g, more typically about 1 g to about 6 g or about 1.5 g to about 5 g) of a vaginal cream as described herein can be furnished in a prefilled container or applicator, for example an applicator similar to that used for Gynazole-1® vaginal cream of KV Pharmaceutical Co., St Louis, Mo. The applicator can be disposable, and more particularly, the applicator can be prefilled with a single unit dose of the composition.

A still further embodiment of the invention provides a method for treating a condition in a female subject for which danazol is indicated. The method is described herein with particular reference to one such condition, endometriosis, but will be understood to be more widely applicable as indicated hereinbelow.

The method comprises administering intravaginally to the subject a therapeutically effective amount of a pharmaceutical composition comprising at least one nonlipoidal internal phase and at least one lipoidal external phase that is bioadhesive to a vaginal mucosal surface, and comprising danazol in an amount of about 1% to about 30%, more typically about 2% to about 30% or about 3% to about 30%, by weight of the composition, wherein upon application of the composition to the vaginal mucosal surface the danazol is released over a period of about 1 to about 10 days.

Such a release period is appropriate for an administration frequency of once every 1 to about 10 days. In some embodiments the composition is administered once to twice weekly, and has a release period is appropriate for such frequency of administration.

Ingredients of the composition, and amounts thereof, can be as described hereinabove. In various embodiments, the composition is administered in an amount containing about 100 mg to about 1000 mg danazol. For example, for twice weekly administration about 100 mg to about 500 mg, and for weekly administration about 200 mg to about 1000 mg, will generally be appropriate. The appropriate dose of danazol is provided by administering a unit dosage amount of the composition, which in the case of a vaginal cream is typically an amount of about 0.5 g to about 10 g, for example about 1 g to about 6 g, most typically about 1.5 g to about 5 g.

Illustratively, a 600 mg/week dosage can be administered by once weekly administration of 3 g of a composition of the invention containing 20% by weight danazol, or by twice weekly administration of 1.5 g of a composition of the invention containing 20% by weight danazol. Greater or lesser dosages can be accommodated by varying the danazol concentration in the composition and/or the amount of the composition administered.

A vaginal cream of the invention can be administered to contact a mucosal surface in the vaginal cavity by means, for example, of an applicator that is optionally pre-filled with a single unit dosage amount of the cream. With the patient optionally in a supine position, the tip of the applicator can be gently inserted high in the vagina, for example in the posterior vaginal fornix, and the cream can be released through the tip by pushing on a plunger of the applicator.

In one embodiment, a method for treating endometriosis in a female subject comprises administering intravaginally to the subject a therapeutically effective amount of a pharmaceutical composition comprising at least one nonlipoidal internal phase and at least one lipoidal external phase that is bioadhesive to a vaginal mucosal surface, and comprising about 20% by weight danazol, about 45% by weight water, about 20% by weight sorbitol, about 8% by weight mineral oil, about 2.8% by weight polyglyceryl-3-oleate, about 2.8% by weight glyceryl monoisostearate, about 0.5% by weight microcrystalline wax and about 1% by weight hydrophobic silica; or a composition substantially bioequivalent thereto.

The terms “treat”, “treating”, “treatment” and the like as used herein can encompass alleviating or reducing severity of a condition or disorder (for example endometriosis, including infertility arising therefrom) and/or alleviating or reducing severity of one or more symptoms of the condition or disorder, for example pain. Administration can be made to a subject currently experiencing symptoms such as pain, or to a subject having the disorder but not currently experiencing symptoms, in anticipation of occurrence of such symptoms. For example, in a subject having endometriosis and susceptible to pain precipitated by menses (dysmenorrhea) or sexual intercourse (dyspareunia), treatment can be administered prior to the precipitating event.

The present method is believed to be particularly effective in cases of pelvic endometriosis, i.e., where ectopic endometrial tissue occurs in tissues of the pelvic region, for example the fallopian tubes and/or ovaries, the outer surface of the uterus and on ligaments supporting the uterus, the lining of the pelvic cavity, other abdominal sites including bladder, bowel, vagina, cervix and vulva, surgical scar tissue, or any combination of the above. In such cases, administration of a composition according to the present method can be effective for alleviation of pain, including non-menstrual pelvic pain, dysmenorrhea and/or dyspareunia.

Administration, for example on a once to twice weekly basis, can continue for as long as necessary to bring the disorder under control and/or alleviate its symptoms. In some embodiments treatment continues for a period of up to 6 months, followed by a period of 6 to 12 months during which danazol therapy is withheld. Suitable treatment periods in individual cases are, illustratively, about 1 week, about 2 weeks, about 1 month, about 3 months or about 6 months.

As mentioned above, any condition for which danazol is indicated can be treated by a method of the invention. Such conditions include inflammatory conditions, more particularly inflammatory conditions occurring in tissues of the pelvic region. Endometriosis is an example of such a condition; others include without limitation pelvic inflammatory disease (PID), interstitial cystitis and vulvovestibulitis.

Other conditions treatable by the present method include autoimmune conditions, more particularly autoimmune connective tissue diseases such as systemic lupus erythematosus, for example where associated with complications such as anemia, e.g., hemolytic anemia; and hematologic conditions such as idiopathic thrombocytopenic purpura or anemias whether or not accompanied by autoimmune disease.

A still further embodiment of the invention provides a contraceptive method. This method comprises administering intravaginally to a female subject a contraceptively effective amount of a pharmaceutical composition comprising at least one nonlipoidal internal phase and at least one lipoidal external phase that is bioadhesive to a vaginal mucosal surface, and comprising danazol in an amount of about 1% to about 30%, more typically about 2% to about 30% or about 3% to about 30%, by weight of the composition, wherein upon application of the composition to the vaginal mucosal surface the danazol is released over a period of about 1 to about 10 days.

Such a method can be used, for example, to provide emergency postcoital contraception when administered postcoitally.

A still further embodiment of the invention provides an endometrial thinning method that can be used pre-operatively. This method comprises administering intravaginally to a female subject prior to surgery an endometrial thinning effective amount of a pharmaceutical composition comprising at least one nonlipoidal internal phase and at least one lipoidal external phase that is bioadhesive to a vaginal mucosal surface, and comprising danazol in an amount of about 1% to about 30%, more typically about 2% to about 30% or about 3% to about 30%, by weight of the composition, wherein upon application of the composition to the vaginal mucosal surface the danazol is released over a period of about 1 to about 10 days.

Such a method is especially useful when administered prior to pelvic surgery, for example hysterectomy, oophorectomy, laparoscopic pelvic surgery, colorectal surgery and/or vaginal surgery.

### EXAMPLES

The following examples are merely illustrative, and do not limit this disclosure in any way.

### Example 1

A danazol vaginal cream, 20% w/w (weight/weight) is prepared having the composition shown in Table 1. USP=United States Pharmacopeia; NF=National Formulary.

TABLE 1
Danazol vaginal cream, 20% w/w

| Ingredient | % w/w |
|---|---|
| water, purified USP | 33.825 |
| sorbitol solution USP¹ | 30.830 |
| edetate disodium USP | 0.050 |
| danazol USP, micronized | 20.000 |
| mineral oil USP | 8.000 |
| polyglyceryl-3-oleate | 2.800 |
| glyceryl monoisostearate | 2.800 |
| microcrystalline wax NF | 0.452 |
| silicon dioxide, hydrophobic | 1.013 |
| methylparaben NF | 0.180 |
| propylparaben NF | 0.050 |
| total | 100.000 |

¹minimum 64% w/w in water

An aqueous phase is prepared by adding water, sorbitol solution and edetate disodium to an appropriately sized stainless steel mixing vessel equipped with an overhead mixer. Mixing is continued until solids are completely dissolved. If needed, temperature is raised to 35-40° C. After cooling if necessary to not higher than 30° C., the resulting aqueous phase is covered and held for further processing (below).

A lipoidal (oil) phase is prepared by adding mineral oil, polyglyceryl-3-oleate, glyceryl monoisostearate, microcrystalline wax and parabens to an appropriately sized stainless steel mixing vessel equipped with an overhead mixer. Temperature is raised during mixing to 70-75° C. to melt and disperse the wax and to dissolve the parabens. With continued mixing, the material is cooled to 30-40° C. and hydrophobic silicon dioxide is added. The resulting oil phase is passed through an appropriately sized mill or disperser and is then covered and held for further processing (below).

While mixing the oil phase at a temperature of approximately 25° C., approximately half of the aqueous phase is added to form a preliminary dispersion. While mixing this preliminary dispersion at a temperature of approximately 25° C., danazol is added and the dispersion is mixed until uniform in appearance.

While mixing the resulting dispersion at a temperature of approximately 25° C., the remaining aqueous phase is added, with mixing until the resulting composition is uniform in appearance. The finished composition is placed in a sealed vessel and held for packaging.

### Example 2

A composition of the present invention, illustratively the composition of Example 1, is administered intravaginally to a female patient having moderate to severe pain associated with endometriosis. The patient is non-pregnant and practicing contraception (for example by use of a spermicide such as nonoxynol-9) and is typically 18 to 45 years of age, with a laparoscopically confirmed diagnosis of endometriosis. At commencement of treatment, she typically has a composite BBSS (Biberoglu & Behrman Sign and Symptom) score >8.0 with a minimum of 4.0 points coming from patient-reported components of dysmenorrhea and non-menstrual pelvic pain. If the patient becomes pregnant she should discontinue treatment.

The composition is typically self-administered, using single-use vaginal applicators, at a twice-weekly or once-weekly frequency, and continues regardless of menses for at least one, more typically at least three menstrual cycles. The danazol dose administered is about 200 to about 1000 mg/week, for example about 400 to about 800 or about 500 to about 700 mg/week. Illustratively, a suitable dose is 600 mg danazol per week, administered for example as 3.0 g of a 20% by weight danazol composition (e.g., the composition of Example 1) once weekly or as 1.5 g of the same composition twice weekly. Twice-weekly dosing is defined as intravaginally administering the composition two times during a calendar week with 3 to 4 days between administrations.

If responsive to the treatment, the patient typically shows at least a 4.0-point improvement over baseline in her composite BBSS score. Of this improvement, at least 3.0 points are typically derived from improvement in patient-reported symptoms of dysmenorrhea, dyspareunia and non-menstrual pelvic pain, including at least 1.0 point from improvement in non-menstrual pelvic pain. Improvement may additionally be evidenced by reduction in use of pain medication for endometriosis pain.

All patents and publications cited herein are incorporated by reference into this application in their entirety.

The words “comprise”, “comprises”, and “comprising” are to be interpreted inclusively rather than exclusively.

1. A pharmaceutical composition comprising at least one nonlipoidal internal phase and at least one lipoidal external phase that is bioadhesive to a vaginal mucosal surface, the composition having an internal/external phase weight ratio of about 50:50 to about 85:15 and comprising danazol in an amount of about 1% to about 30% by weight of the composition, wherein danazol is present in micronized form or in nanoparticulate form and upon application of the composition to the vaginal mucosal surface the danazol is released over a period of about 1 to about 10 days.

2. The composition of claim 1, wherein the danazol is present in an amount of about 3% to about 30% by weight of the composition.

3. The composition of claim 1, wherein the danazol is present in an amount of about 5% to about 25% by weight of the composition, and upon application of the composition to the vaginal mucosal surface, the danazol is released over a period of about 2 to about 8 days.

4. The composition of claim 1, wherein the danazol is released over a period consistent with a once to twice weekly dosing schedule.

5. The composition of claim 1, in a form of a vaginal cream or a suppository that melts or softens at body temperature to form a cream.

6. The composition of claim 1, wherein the internal phase is aqueous and comprises at least one pharmaceutically acceptable polyol.

7. The composition of claim 6, wherein the at least one polyol comprises glycerol, one or more glycols and/or one or more sugar alcohols.

8. The composition of claim 6, wherein the internal phase comprises no vaginal irritant amount of propylene glycol.

9. The composition of claim 6, wherein the at least one polyol comprises sorbitol.

10. The composition of claim 1, wherein the external phase comprises at least one pharmaceutically acceptable oil.

11. The composition of claim 10, wherein the at least one oil comprises a mineral oil and/or a vegetable oil.

12. The composition of claim 10, wherein the at least one oil comprises a mineral oil having a viscosity of about 25 to about 65 centistokes.

13. The composition of claim 10, wherein the external phase further comprises at least one pharmaceutically acceptable emulsifying agent.

14. The composition of claim 13, wherein the at least one emulsifying agent comprises one or more medium and/or long chain monoglycerides and/or diglycerides, and/or one or more polyglyceryl esters of fatty acids.

15. The composition of claim 13, wherein the at least one emulsifying agent comprises glyceryl monoisostearate and/or polyglyceryl-3-oleate.

16. The composition of claim 10, wherein the external phase further comprises at least one pharmaceutically acceptable viscosity modulating agent.

17. The composition of claim 16, wherein the at least one viscosity modulating agent comprises microcrystalline wax and/or hydrophobic silica.

18. A pharmaceutical composition comprising at least one nonlipoidal internal phase and at least one lipoidal external phase that is bioadhesive to a vaginal mucosal surface, and comprising about 20% danazol, wherein danazol is present in micronized form or in nanoparticulate form and wherein the internal phase comprises about 45% water and about 20% sorbitol and the external phase comprises about 8% mineral oil, about 2.8% polyglyceryl-3-oleate, about 2.8% glyceryl monoisostearate, about 0.5% microcrystalline wax and about 1% hydrophobic silica, all percentages being by weight of the composition as a whole; or a composition substantially bioequivalent thereto when administered intravaginally.

19-24. (canceled)

25. A vaginal danazol delivery system comprising (a) a pharmaceutical composition comprising at least one nonlipoidal internal phase and at least one lipoidal external phase that is bioadhesive to a vaginal mucosal surface, and comprising danazol in an amount of about 1% to about 30% by weight of the composition, wherein upon application of the composition to the vaginal mucosal surface the danazol is released over a period of about 1 to about 10 days; and (b) an applicator.

26. The delivery system of claim 25, wherein the composition comprises danazol in an amount of about 3% to about 30% by weight.

27-54. (canceled)
